(19) United States
(12) Patent Application Publication
(10) Pub. No.: US 2023/0386691 A1
(43) Pub. Date: Nov. 30, 2023

(54) COMPOSITION FOR RADIATION SHIELD USING MICROORGANISMS AND RADIATION SHIELD MATERIAL INCLUDING THE SAME

(71) Applicant: COENBIO CO., LTD., Seongnam-si (KR)

(72) Inventors: Kyu Jin YUM, Anyang-Si (KR); Jung A LEE, Seoul (KR)

(21) Appl. No.: 18/031,874

(22) PCT Filed: Nov. 23, 2020
(86) PCT No.: PCT/KR2020/016629

§ 371 Date: Apr. 13, 2023

(30) Foreign Application Priority Data
Oct. 14, 2020 (KR) 10-2020-0132329

(51) Int. Cl. G21F 1/10; C12N 1/14; C12N 11/04; G21F 3/02

(52) U.S. Cl. CPC G21F 1/10; C12N 1/14; C12N 11/04; G21F 3/02

(57) ABSTRACT

A composition for radiation shield includes one or more microorganisms selected from the group consisting of Cladosporium sp. microorganism, Phanerochaete sp. microorganism and Trichosporon sp. Microorganism, and a radiation shield material includes the composition for radiation shield.

### CROSS REFERENCE TO RELATED APPLICATIONS

This application is a National Stage Entry of International Patent Application No. PCT/KR2020/016629, filed on Nov. 23, 2020, which claims priority from and the benefit of Korean Patent Application No. 10-2020-0132329, filed on Oct. 14, 2020, each of which is hereby incorporated by reference for all purposes as it fully set forth herein.

### BACKGROUND

### Field

Embodiments of the inventions relate generally to a composition for radiation shield using microorganisms and as radiation shield material including the composition.

### Discussion of the Background

Radioactive ray is generally an electromagnetic wave or mrtiele beam generated when a radioactive material (radioactive isotope) decays into a stable material, and gamma ray (γ-ray), X-ray, neutron ray (neutron), beta-ray (β-ray), alpha-ray (α-ray) and the like fall within this category.

Radioactive ray is mainly generated in places where radioactive materials are handled, such as nuclear medicine-related hospitals, nuclear power plants, nuclear waste treatment facilities, military nuclear material facilities, and non-destructive industries. When exposed to a large amount of these radiations or exposed to the radiation for a long period of time, DNA is damaged and is fatal to the human body, so effective radiation shielding is a very important factor for workers.

The handling of radioactive materials is increasing year by year, and the global radiation shielding-related market is currently known to be in about 1,700 trillion won scale.

Radiation shield materials have long been manufactured using heavy metal powders such as lead or lead compounds. Even now, most radiation shield materials contain still a large amount of lead. However, lead dust components not only are harmful to the human body, but also cause environmental pollution due to heavy metal waste. Therefore, the use of heavy metal products such as lead is regulated mainly in the United States, Europe and the like. In addition, the shield material made of lead is not only heavy, but also uncomfortable to wear, which limits the activity of workers, and manufacturing cost is very high.

In connection with lead-free radiation shield materials, Korean Patent Registration No. 10-1145703 discloses a radiation shield sheet manufactured by mixing and curing a silicone polymer, tourmaline, barium sulfate and a silicone curing accelerator.

Korean Patent Registration No. 10-2035512 discloses a coating agent for radiation shield in which a radiation shielding agent such as tungsten, bismuth, barium, europium, dysprosium, and strontium is mixed with a binder resin such as epoxy or urethane, but there is still a problem of environmental pollution because of using heavy metals as a shielding agent, and the materials used are also expensive.

Korean Patent Registration No. 10-1731785 discloses a hydrogel matrix in which a first polymer such as alginate, chitosan, hyaluronic acid and a second polymer such as polyacrylamide, polyvinyl alcohol, and polyethylene are cross-linked in order to impart ductility and elasticity to a radiation shield material, but metal particles such as boron, lithium, gadolinium, samarium, europium cadmium, dysprosium, lead, iron, and tungsten are used materials for shielding radiation.

Although the above-mentioned prior arts contain a polymer material for radiation shield materials, components for shielding radiation are substantially dependent on heavy metals and may still be harmful to the human body or the environment.

The above information disclosed in this Background section is only for understanding of the background of the inventive concepts, and, therefore, it may contain information that does not constitute prior art.

### SUMMARY

It is an object of the present invention to provide a composition for radiation shield and a radiation shield material that have excellent radiation shielding ability, are harmless to the human body, has light weight even without environmental pollution, is comfortable to wear, and can be manufactured at a low price.

The present invention provides a composition for radiation shield including one or more microorganisms selected from the group consisting of Cladosporium sp. microorganism, Phanerochaete sp. microorganism and Trichosporon sp. microorganism.

In one embodiment, Cladosporium sp. microorganism may be Cladosporium cladosporioides, and in one example, Cladosporium cladosporioides Ceb-RadF-001 strain (accession number: KCCM12440P) can be used.

In one embodiment, the Phanerochaete sp. microorganism may be Phanerochaete chrysosporium or Phanerochaete sordida, and in one example, Phanerochaete chrysosporium) Y-2 strain (accession number: KCCM10725P) can be used.

In one embodiment, the Trichosporon sp. microorganism may be Trichosporon loubieri, and in one example, Trichosporon loubieri Y1-A strain (accession number: KCTC10876BP) can be used.

Meanwhile, the present invention provides a radiation shield material including the composition for radiation shield.

In one embodiment, the radiation shield material may include a microorganism immobilized media.

The microorganism immobilized media may be a porous material, but is not limited thereto.

The microorganism immobilized media may be selected from polymer resin, activated carbon, zeolite, non-woven fabric or woven fabric, but is not limited thereto.

The microorganism immobilized media may further include carbon nanotubes or graphene.

The microorganism of the composition for radiation shield according to the present invention not only has strong resistance to radiation, but also absorbs radiation and grows using it as growth energy, so the radiation shielding effect is very excellent, and the shielding effect is comparable to or more excellent than shield materials made of lead.

Further, since the radiation shield material according to the present invention does not use lead or other metal powder as a material, it is harmless to the human body and has no problem of environmental pollution even at the disposal. Compared to conventional shield materials, it is remarkably lighter and more flexible, and is comfortable for the activity of workers, so that it has high utility value as a material or a construction material for radiation shielding clothing, aprons, gloves, shoes and the like.

It is to be understood that both the foregoing general description and the following detailed description are illustrative and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate illustrative embodiments of the invention, and together with the description serve to explain the inventive concepts.

FIG. 1 shows the arrangement of microbial samples according to the irradiation distance in the radiation (gamma ray) irradiating room carried out in the present invention.

FIG. 2 shows the change of each microbial strain with an increase in adiation (gamma ray) dose in the experimental example of the present invention.

FIG. 3 (a) is a photograph of the growth of a Cladosporium cladosporioides strain to which radioactive rays are not irradiated, and (b) is a photograph of the growth of a Cladosporium cladosporioides strain having an absorbed dose of 392 Gy.

FIG. 4 shows a porous polyurethane filter used for attaching microorganisms of the present invention.

FIG. 5 shows the arrangement of microbial samples according to the irradiation distance in the radiation (gamma ray) irradiating room carried out in the present invention.

FIG. 6 shows a radiation shielding rate measuring mechanism, in which (a) shows a radiation shielding rate measuring device, and (b) shows a radiation shielding rate measuring sensor.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing illustrative features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

As used herein, the term ‘radiation’ includes ionizing radiation, and includes gamma rays, X-rays, neutron rays, alpha rays, and beta rays.

As used herein, the term ‘microorganism’ is ‘strain’ or includes ‘strain’ unless otherwise stated.

As used herein, the term “single microorganism” means a microorganism composed of only one strain.

As used herein, the term “complex microorganism” means a group of microorganisms consisting of two or more kinds of strains. The complex microorganisms include those obtained by mixing a culture solution of a single microorganism or culturing two or more kinds of strains in the same medium.

As used herein, the term ‘low dose’, ‘medium dose’ and ‘high dose’ related to radiation are only a term used in the examples of the present invention to compare the radiation shielding effects according to the magnitude of the radiation dose with each other, and do not mean the absolute range of toxicity. The dose unit ‘Gy’ (Gray) is a unit representing the radiation energy absorbed by a material, and when 1 joule (J) per 1 kg of material is absorbed, it is defined as 1 Gy. For example, when exposed to 1 Gy or more of radiation at a time, acute radiation sickness appears in almost everyone. If systemic exposure doses are extremely high (20 Gy or more), severe acute neurovascular disease can occur. For reference, the highest radiation exposure dose of power plant workers in the Chernobyl accident was 16 Gy. In the examples of the present invention, 220.6 Gy and 392 Gy are expressed as low doses, but this is relatively low compared to 21,687 Gr and 38,600 Gy and is only expressed, which is equivalent to tens to hundreds of times the lethal dose.

The present inventors have studied various microorganisms in order to select microorganisms capable of effectively blocking high-risk radiation such as gamma rays and X-rays, and as a result, confirmed that the radiation shielding ability was remarkably excellent in three types of microorganisms.

The present invention provides a composition for radiation shield including one or more microorganisms selected from the group consisting of Cladosporium sp. microorganism, Phanerochaete sp. microorganism and Trichosporon sp. microorganism.

The composition for radiation shield of the present invention may include the above strains alone (single microorganism), or may include as a mixture of the above strains (complex microorganism).

The composition for radiation shield may be the strain itself, or a culture solution or a mixed solution thereof, and the strain, culture solution, and mixed solution may be in a semi-dried or dried form as necessary.

The Cladosporium sp. Microorganism is known as black fungi that produce melanin pigments, and Cladosporium cladosporioides is preferable, but is not limited thereto. In one example, it may be a Cladosporium cladosporioides Ceb-RadF-001 strain. The Cladosporium cladosporioides Ceb-RadF-001 strain was deposited at the Korean Culture Center of Microorganisms under the accession number KCCM12440P on Feb. 26, 2019.

The Phanerochaete sp. Microorganism is known as white fungi, and Phanerochaete chrysosporium or Phanerochaete sordida is preferable, but is not limited thereto. In one example, Phanerochaete sp. Y-2 strain can be used. The Phanerochaete sp. Y-2 strain was deposited at the Korean Culture Center of Microorganisms under the accession number KCCM10725P on Feb. 26, 2019.

The Trichosporon sp. microorganism is yeast, and Trichosporon loubieri can be preferably used, but is not limited thereto. In one example, Trichosporon loubieri Y1-A strain can be used, The Trichosporon loubieri Y1-A strain was deposited at the Korean Culture Center of Microorganisms under the accession number KCCM10876BP on Feb. 26, 2019.

The microorganisms according to the present invention can be cultured in a medium. Natural medium or synthetic medium may be used as the culture medium. The culture medium may include a carbon source, at nitrogen source, and inorganic salts.

The carbon source of the culture medium is not limited, but can be carbon sources known in the field of microbial culture, for example, sugars such as glucose, sucrose, fructose, maltose, lactose, dextrin, dextrose, starch, organic acids such as malic acid and citric acid, fatty acids having a low molecular weight, and the like.

The nitrogen source of the culture medium is not limited, but can be nitrogen sources known in the field of microbial culture, for example, peptone, meat extract, yeast extract, dried yeast, casein, whey protein, soybean, ammonium salt, nitrate and other organic or inorganic nitrogen, sulfur-containing compounds and the like.

The inorganic salt of the culture medium is not limited, but can be inorganic salts known in the field of microbial culture, for example, magnesium (Mg) manganese (Mn), calcium (Ca) iron (Fe), potassium (K), sodium (Na), phosphorus (P), sulfur (S), boron (B), molybdenum (Mo), copper (Cu), cobalt (Co), zinc (Zn) and the like.

The culture medium may further contain growth factors, if necessary, in addition to components of a carbon source, a nitrogen source and an inorganic salt. The growth factor may be amino acid, vitamin, nucleic acid, or compounds related thereto.

The microorganisms according to the present invention are not limited, but may be cultured in a temperature range 20° C. to 40° C., preferably in as culture temperature range of 25° C. to 35° C.

The microorganism according to the present invention is not limited, but may be cultured for 12 hours to 7 days, preferably for 12 hours to 5 days.

The composition for radiation shield of the present invention may include a single microorganism or a complex microorganism. The complex microorganism of the present invention may be prepared bye mixing a culture solution obtained by culturing each strain individually, or may be prepared by culturing two or more strains together.

The concentration of microorganisms in the composition for radiation shield of the present invention is not limited, but the concentration may be 0.5×10² CFU/ml or more, preferably 0.5×10³ CFU/ml or more, more preferably 0.5×10⁴ CFU/ml or more, and still more preferably 0.5×10⁵ CFU/ml or more.

In one embodiment, the composition for radiation shield of the present invention may be a culture medium of microorganisms or a microorganism concentrated or purified from the culture medium, and may be in a dry or semi-dried form, if necessary.

The composition for radiation shield of the present invention may contain an acceptable carrier, if necessary. The acceptable carriers are those suitable as a nutrient for the selected microorganisms. For example, physiological saline, sterile water, buffered saline, dextrose solution, maltodextrin solution, glycerol, and one or more of these components may be mixed and used. Other conventional additives such as antioxidants, buffers, and bacteriostatic agents may be added as needed. In addition, a diluent, a dispersant, a surfactant, a binder, and a lubricant may be additionally added to form a liquid formulation such as an aqueous solution, a suspension, an emulsion, or a solid formulation such as a powder.

Meanwhile, the present invention provides a radiation shield material including the composition for radiation shield. The radiation shield material of the present invention may include a microorganism immobilized media. The microbial immobilized media can be used for the purpose of maintaining the shape of a shield material while allowing microorganism to attach well. In one embodiment, the microorganism-immobilized media may be a porous material such as a polymer resin in a matrix form having pores, activated carbon, zeolite, nonwoven fabric, woven fabric, and the like. The polymer resin is not limited, but may be, for example, synthetic polymers such as polyurethane, polyacrylamide, polyethylene glycol, and polyvinyl alcohol, or natural polymers such as agar, agarose, k-carrageenan, alginate, and chitosan. The shape of the microbial immobilized media is not limited, but may be foam, sponge, filter, sheet, or film, and if necessary, it may be in powder or pellet form. In one embodiment, the composition for radiation shield may be supported on a microorganism-immobilized media to stabilize attachment, and if necessary, additional cultivation can be performed. In addition, it may be semi-dried or dried, if necessary.

In one embodiment, the radiation shield material according to the present invention may be prepared by impregnating a microorganism-immobilized media in the composition for radiation shield (microbial culture solution or concentrate). The impregnation time is to ensure that microorganisms are sufficiently attached to the media, and it can be appropriately adjusted according to the type of microorganism and media. The impregnation time may be 12 hours or more, but is not limited thereto. The impregnation time may be preferably for 1 to 15 days. If necessary, a semi-drying or drying process may be added after impregnation. Drying is preferably natural drying.

In one embodiment, the microorganism immobilized media may be prepared by a known entrapment method or encapsulation method.

The microorganism immobilized media may further include carbon nanotubes or graphene that is known to have a radiation shielding effect.

The composition for radiation shield or a radiation shield material according to the present invention can be used in various ways as materials such as clothing for radiation shielding (shield clothing), shield aprons, shield gloves, shield hats, shield shoes, shield sheets, and shield panels. At this time, the composition for radiation shield or a radiation shield material may be used as an inner material of a shielding product.

Hereinafter, the present invention will be described in more detail with reference to examples. However, these examples are for illustrative purposes only, and the scope of the present invention is not limited thereto.

### Example 1: Production of Composition for Radiation Shield Containing Single Microorganism (Phanerochaete Chrysosporium Strain)

Phanerochaete chrysosporium Y-2 strain (accession number: KCCM10725P) was cultured in a BBM medium (Bold's basal medium to which KH₂PO₄ 0.175 g, CaCl₂, 2H₂O 0.025 g, MgSO₄·7H₂O 0.075 g, NaNO₃ 0.25 g, K₂HPO₄ 0.075 g, NaCl 0.025 g, Na₂EDTA 0.1 g, KOH 0.062 g, FeSO₄·7H₂O 0.0498 g, H₃BO₃ 0.115 g, MnCl₂·4H₂O 0.00181 g, ZnSO₄·7H₂O 0.000222 g, NaMoO₄·5H₂O 0.00039 g, CuSO₄·5H₂O 0.000079 g, Co(NO₃)₂·6H₂O 0.0000494 g were respectively added to 1 liter of purified water.

### Example 2: Production of Composition for Radiation Shield Containing Single Microorganism (Trichosporon Loubieri Strain)

Trichosporon loubieri Y1-A strain (accession No.: KCTC10876BP) was cultured in a PDB medium to which 200 g of potato infusion and 20 g of dextrose was respectively added to 1 liter of purified water, by using a shaking incubator at 130 rpm and 30° C. for 20 hours. Culturing was stopped when the microbial growth curve was in the middle of the logarithmic phase and the normal phase, and was used for subsequent experiments.

### Example 3: Production of Composition for Radiation Shield Containing Single Microorganism (Cladosporium Cladosporioides Strain)

Cladosporium cladosporioides Ceb-RadF-001 strain (accession No.: KCCM12440P) was cultured in a NB(nutrient broth) medium to which 3 g of beef extract, 5 g of peptone, and 8 g of NaCl were added to 1 liter of purified water, by using a shaking incubator at 130 rpm and 30° C. for 20 hours. Culturing was stopped when the microbial growth curve was in the middle of the logarithmic phase and the normal phase, and was for subsequent experiments.

### Example 4: Production of Composition for Radiation Shield Containing Two Kinds of Microorganisms

Phanerochaete chrysosporium Y-2 strain culture solution produced in Example 1 and Trichosporon loubieri Y1-A strain culture solution produced in Example 2 were mixed in a ratio of 1:1 to prepare a two-kind complex microorganism culture solution.

### Example 5: Production of Composition for Radiation Shield Containing Two Kinds of Microorganisms

Phanerochaete chrysosporium Y-2 strain culture solution produced in Example 1 and Cladosporium cladosporioides Ceb-RadF-001 strain culture solution produced in Example 3 were mixed in a ratio of 1:1 to prepare a two-kind complex microorganism culture solution.

### Example 6: Production of Composition for Radiation Shield Containing Two Kinds of Microorganisms

Trichosporon loubieri Y1-A strain culture solution produced in Example 2 and Cladosporium cladosporioides Ceb-RadF-001 strain culture solution produced in Example 3 were mixed in a ratio of 1:1 to prepare a two-kind complex microorganism culture solution.

### Example 7: Production of Composition for Radiation Shield Containing Three Kinds of Microorganisms

Phanerochaete chrysosporium Y-2 strain culture solution produced in Example 1, Trichosporon loubieri Y1-A strain culture solution produced in Example 2 and Cladosporium cladosporioides Ceb-RadF-001 strain culture solution produced in Example 3 were mixed in a ratio of 1:1:1. to prepare a three-kind complex microorganism culture solution.

### Experimental Example 1: Measurement of the Survival Rate of Microbial Strains According to Radiation Irradiation

### (1) Preparation of Sample

200 mL of each of the microorganism-containing compositions prepared in Examples 1 to 3 was added to a 0.5 L Erlenmeyer flask having a baffle, and microbial samples were prepared by closing with a hydrophobic silicon stopper having air permeability and a small amount of water evaporation. Three microbial samples (3 repetitions) were made by the kinds of microorganims.

### (2) Radiation Irradiation

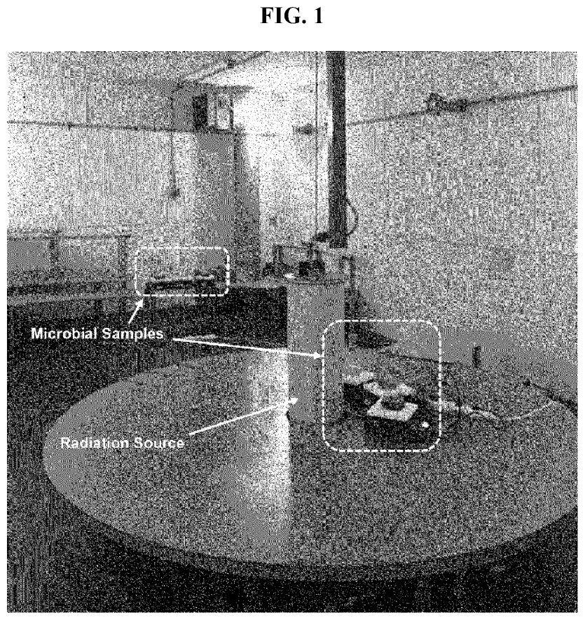

As shown in FIG. 1, the microbial samples were located at a distance of 25 cm and 379 cm, respectively, from a radiation source in a cobalt 60 (⁶⁰Co) radiating room, placed on a shake (DA1HAN Scentific model SHO-2D) and irradiated with radioactive ray while continuously shaking at a speed of about 100 RPM. The control was not irradiated.

The temperature of the laboratory during the experiment was maintained in the range of 21° C. to 25° C. without any artificial control. Mainly, the fluorescent lamp in the laboratory was turned on during business hours, and remained turned off after work ending time.

The absorbed dose rate of the sample was calculated from the radiation source of ⁶⁰Co and the irradiation distance, and radioactive ray was irradiated for about 5 days for 93.08 hr.

The ⁶⁰Co radiation dose conditions are shown in Table 1 below.

TABLE 1
Absorbed dose rate and total absorbed dose

| | Irradiation | Irradiation | Absorbed dose |
|---|---|---|---|
| Absorbed dose rate | distance | time | (D) |
| 233 Gy/hr | 25 cm | 93.08 hr | 233 Gy/hr × 93.08 |
| | | | hr = 21,687 Gy |
| 2.37 Gy/hr | 379 cm | 93.08 hr | 2.37 Gy/hr × 93.08 |
| | | | hr = 220.6 Gy |

### (3) Measuring the Survival Rate of Microorganisms After Irradiation

For Trichosporon loubieri strain, the viable cell count (bacterial concentration) of the microorganism was measured before irradiation (0 days) and after irradiation for about 5 days, respectively. For Cladosporium cladosporioides strain and Phanerochaete chrysosporium strain, the amount of microbial biomass was measured before irradiation (0 days) and after irradiation for about 5 days. The results are shown in Tables 2 to 4 below.

The survival rate of microorganisms was calculated as a percentage by using the strain cultured for about 5 days without irradiation (0 Gy) as a control and calculating the viable cell count (bacterial concentration) or the biomass amount of strain according to the radiation dose compared to the control.

TABLE 2
Survival rate of Cladosporium cladosporioides
strain according to Co-60 radiation dose

| | Dry biomass (mg/100 mL) | | Survival rate (%) compared to |
|---|---|---|---|
| Dose | 0 day | 5 day | control on the day 5 |
| 0 Gy | 205 | 495 | 100% |
| (Control) | | | |
| 220.6 Gy | 205 | 531 | 107.3% |
| 21687 Gy | 205 | 289 | 58.4% |

TABLE 3
Survival rate of Phanerochaete chrysosporium
strain according to Co-60 radiation dose

| | Dry biomass (mg/100 mL) | | Survival rate (%) compared to |
|---|---|---|---|
| Dose | 0 day | 5 day | control on the day 5 |
| 0 Gy | 288 | 623 | 100% |
| (Control) | | | |
| 220.6 Gy | 288 | 588 | 94.4% |
| 21687 Gy | 288 | 290 | 46.5% |

TABLE 4
Survival rate of Trichosporon loubieri
strain according to Co-60 radiation dose

| | Viable cell count (CFU/mL) | | Survival rate (%) compared to |
|---|---|---|---|
| Dose | 0 day | 5 day | control on the day 5 |
| 0 Gy | 1.0 × 10⁶ | 7.1 × 10⁶ | 100% |
| (Control) | | | |
| 220.6 Gy | 1.0 × 10⁶ | 6.9 × 10⁶ | 97.2% |
| 21687 Gy | 1.0 × 10⁶ | 5.6 × 10⁴ | 0.8% |

As shown in Tables 2 to 4, Phanerochaete chrysosporium strain and Trichosporon loubieri strain showed a high survival rate of more than 94% at 220.6 Gy, and Cladosporium cladosporioides strain showed a growth of 7.3% or more compared to the control. This is judged that the Cladosporium cladosporioides strain showed higher growth rate by using high energy of radiation as a growth energy source. Phanerochaete chrysosporium strain and Cladosporium cladosporioides strain survived 46.5% and 58.4%, respectively, even after absorbing high doses (21.687 Gy). For reference, it can be seen that Deinococcus radiodurans strain, which the US researchers reported as being very resistant to radioactivity, showed a 1% survival rate at 9,000 Gy, whereas the microorganisms of the present invention have remarkably higher viability against radiation.

### Experimental Example 2: Morphological Change Experiment of Microorganisms According to Radiation Dose

The experiment was carried out in the same manner as in Experimental Example 1, but the irradiation was carried out by increasing the dose to 392 Gy, 3,810 Gy, and 38,600 Gy, respectively, for 7 days. In order to confirm the morphological change of microbial strains according to the irradiation dose, samples collected by 10 ml were diluted 10⁴ times with sterile physiological saline, acid microbial strains that survived and grown after being placed on solid medium (NA, TSA, PDA) containing nutrients for each strain were observed and shown in FIG. 2.

As shown in FIG. 2, it can be confirmed that the higher the dose, the larger the thickness and size of the mycelium and spore, and endoplasmic reticulum and nuclear (DNA) substances increased in the mycelium and spores.

### Experimental Example 3: Measurement of the Growth Rate of Cladosporium Cladosporioides Strain According to the Radiation Dose

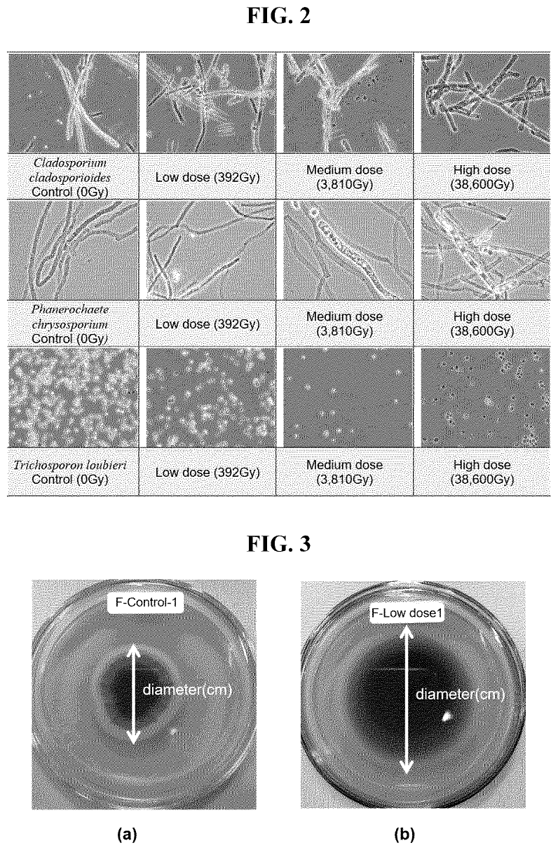

For the Cladosporium cladosporioides strain having an increased growth rate even at 220.6 Gy, the bacterial growth rate was measured again while irradiating gamma rays at 392 Gy, 3810 Gy, and 38600 Gy, respectively, for 7 days, and the results are shown in Table 5 and FIG. 3 below. Control is a strain that has not been irradiated with radiation.

TABLE 5
Measurement result of bacterial growth rate
after irradiation with Co-60 for 7 days

| Irradiation days | 0 day | 7 day |
|---|---|---|
| Control | Diameter 3.60 cm | Diameter 3.90 cm |
| Irradiated | Diameter 3.59 cm | Diameter 5.20 cm (1.78 times |
| at 392 Gy | | increase in bacterial |
| | | growth rate compared |
| | | to control) |
| Irradiated at | Diameter 3.61 cm | Diameter 3.10 cm |
| 3,810 Gy | | |
| Irradiated at | Diameter 3.62 cm | Diameter 3.05 cm |
| 38,600 Gy | | |

As shown in FIG. 3 and Table 5, it can be confirmed that Cladosporium cladosporioides strain has a growth diameter of 5.20 cm at a dose of 392 Gy, which is 1.78 times higher than that of the control. It can also be seen that it does not die even at a high dose of 38,600 Gy.

### Example 8: Preparation of Radiation Shield Material

As shown in FIG. 4, the microorganism-containing composition for radiation shield prepared in Examples 1 to 7 was supported on a porous polyurethane filter having a void of 25 ppi and a thickness of 10 mm, respectively, and then dried to produce a radiation shield material sample to which each microorganism was attached.

### Experimental Example 4: Experiment of Radiation Shielding Effect

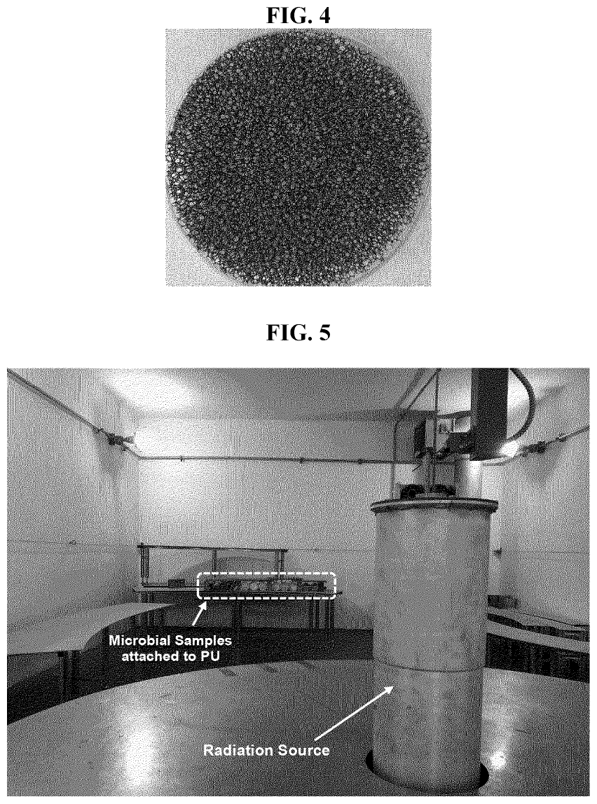

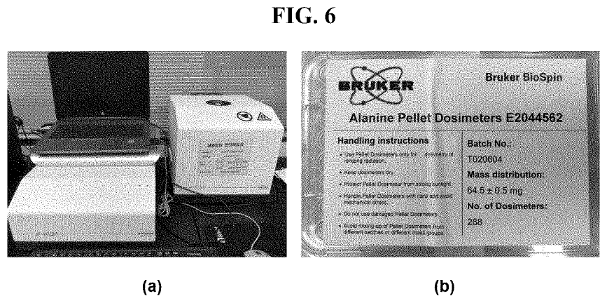

In order to measure the radiation shielding effect of the microbial strains according to the present invention, the radiation shield material sample prepared in Example 8 was placed in a ⁶⁰Co radiation irradiating room as shown in FIG. 5, and irradiated with radioactive rays under the irradiation conditions for shielding ability tests shown in Table 6 below. The shielding rate measurement results are shown in Table 7 below. At this time, the radiation shielding rate measurement sensor was attached to the back of the polyurethane shielding sample using Alanine Pellet Dosimeters (Bruker BioSpin, USA), and the measurement equipment was e-scan (Bruker BioSpin, USA) (FIG. 6).

TABLE 6
Irradiation condition for experimentation
of radiation shielding ability

| | | |
|---|---|---|
| Radiation irradiation | Co-60 | Temperature: 22.7 ± 1° C. |
| | | (irradiation room) |
| Absorbed dose | 2.3 | Light illuminance: |
| rate (Gy/hr) | | 30-70 Lx |

| | |
|---|---|
| Total irradiation time | 48 hr |

Total absorbed dose rate: 110.4 Gy

TABLE 7
Measurement result of radiation shielding rate

| | | | Radiation Shielding Rate (%) | | |
|---|---|---|---|---|---|
| | | | [(D0 − D)/D0)]×100 | | |
| | Treated group (3 repetitions) | | | | Shielding rate |
| Example | Strain | | 22 hr | 48 hr | average(%) |
| Control | — | 1 repetition | 7.714206 | 10.222980 | |
| | | 2 repetition | 5.359606 | 7.970050 | |
| | | 3 repetition | 7.847337 | 9.021764 | |
| | | Average value | 6.973717 | 9.071598 | 8.2 |
| Example 1 | P. chrysosporium | 1 repetition | 15.006447 | 14.156591 | |
| | | 2 repetition | 14.716102 | 14.255646 | |
| | | 3 repetition | 14.467462 | 14.714976 | |
| | | Average value | 14.730004 | 14.375738 | 14.6 |
| Example 2 | T. loubieri | 1 repetition | 15.251054 | 15.172152 | |
| | | 2 repetition | 12.580364 | 10.388004 | |
| | | 3 repetition | 12.132538 | 13.717625 | |
| | | Average value | 13.321318 | 13.092594 | 13.2 |
| Example 3 | C. cladosporioides | 1 repetition | 28.317547 | 25.219327 | |
| | | 2 repetition | 21.776832 | 22.316921 | |
| | | 3 repetition | 22.431189 | 19.726522 | |
| | | Average value | 24.175189 | 22.420923 | 23.3 |
| Example 4 | P. chrysosporium + | 1 repetition | 17.034614 | 14.978545 | |
| | T. loubieri | 2 repetition | 15.698547 | 15.125686 | |
| | | 3 repetition | 15.495756 | 15.354685 | |
| | | Average value | 16.076306 | 15.152972 | 15.6 |
| Example 5 | P. chrysosporium + | 1 repetition | 32.546856 | 25.969869 | |
| | C. cladosporioides | 2 repetition | 25.456256 | 24.987895 | |
| | | 3 repetition | 24.966856 | 23.546686 | |
| | | Average value | 27.656656 | 24.834817 | 26.2 |
| Example 6 | T. loubieri + | 1 repetition | 30.569624 | 26.594856 | |
| | C. cladosporioides | 2 repetition | 27.695614 | 24.496256 | |
| | | 3 repetition | 25.266562 | 25.765922 | |
| | | Average value | 27.843933 | 25.619011 | 26.7 |
| Example 7 | P. chrysosporium + | 1 repetition | 30.469856 | 26.995586 | |
| | T. loubieri + | 2 repetition | 27.456253 | 25.262575 | |
| | C. cladosporioides | 3 repetition | 27.685794 | 26.096846 | |
| | | Average value | 28.537301 | 26.118336 | 27.3 |

| | | | |
|---|---|---|---|
| Thickness 2 mm lead | 13.668271 | 11.646847 | 12.7 |
| Thickness 4 mm lead | 17.361586 | 19.719506 | 18.5 |
| Thickness 6 mm lead | 29.168030 | 26.541714 | 27.9 |

As shown in Table 7 above, treated groups (Examples 1 to 3) to which the single microorganisms according to the present invention were attached showed a higher radiation shielding rate than that of the control and the 2 mm thick lead. In particular, the treated group to which Cladosporium cladosporioides strain was attached showed the highest radiation shielding rate of 23.3%, which showed a higher shielding rate than that of 4 mm thick lead. In addition, the treated group to which the complex microorganism according to the present invention was attached showed a significantly higher radiation shielding rate compared to the single microorganism, and the compositions of Examples 5 to 7 showed a radiation shielding rate comparable to that of 6 mm thick lead.

### INDUSTRIAL APPLICABILITY

The present invention relates to a composition for radiation shield using microorganisms and a radiation shield material comprising the composition.

| | | |
|---|---|---|
| 0-1 | Form PCT/RO/134 | |
| | Indications Relating to | |
| | Deposited | |
| | Microorganism(s) or | |
| | Other Biological Material | |
| | (PCT Rule 13 bis) | |
| 0-1-1 | Prepared Using | ePCT-Filing |
| | | Version 4.7.111 MT/FOP 20201118/0.20.5.24 |
| 0-2 | International Application | PCT/KR2020/016629 |
| | No. | |
| 0-3 | Applicant's or agent's file | SOP-20012-CO |
| | reference | |
| 1 | The indications made | |
| | below relate to the | |
| | deposited | |
| | microorganism(s) or other | |
| | biological material | |
| | referred to in the | |
| | description on: | |
| 1-1 | Paragraph number | 37 |
| 1-3 | Identification of deposit | |
| 1-3-1 | Name of depositary | KCCM Korean Culture Center of |
| | institution | Microorganisms |
| 1-3-2 | Address of depositary | Korean Culture Center of Microorganisms |
| | institution | (KCCM) |
| | | 361-221, Yurim B/D |
| | | Honje 1 |
| | | Sudaemun |
| | | Seoul 120-091 |
| | | Republic of Korea |
| 1-3-3 | Date of deposit | 26 Feb. 2019 (26 Feb. 2019) |
| 1-3-4 | Accession Number | KCCM 12440P |
| 1-4 | Additional Indications | |
| 1-5 | Designated States for | All designations |
| | Which Indications are | |
| | Made | |
| 1-6 | Separate Furnishing of | |
| | Indications | |
| | These indications will be | |
| | submitted to the | |
| | International Bureau later | |
| 2 | The indications made | |
| | below relate to the | |
| | deposited | |
| | microorganism(s) or other | |
| | biological material | |
| | referred to in the | |
| | description on: | |
| 2-1 | Paragraph number | 38 |
| 2-3 | Identification of deposit | |
| 2-3-1 | Name of depositary | KCCM Korean Culture Center of |
| | institution | Microorganisms |
| 2-3-2 | Address of depositary | Korean Culture Center of Microorganisms |
| | institution | (KCCM) |
| | | 361-221, Yurim B/D |
| | | Honje 1 |
| | | Sudaemun |
| | | Seoul 120-091 |
| | | Republic of Korea |
| 2-3-3 | Date of deposit | 19 Dec. 2005 (19 Dec. 2005) |
| 2-3-4 | Accession Number | KCCM 10725P |
| 2-4 | Additional Indications | |
| 2-5 | Designated States for | All designations |
| | Which Indications are | |
| | Made | |
| 2-6 | Separate Furnishing of | |
| | Indications | |
| | These indications will be | |
| | submitted to the | |
| | International Bureau later | |
| 3 | The indications made | |
| | below relate to the | |
| | deposited | |
| | microorganism(s) or other | |
| | biological material | |
| | referred to in the | |
| | description on: | |
| 3-1 | Paragraph number | 39 |
| 3-3 | Identification of deposit | |
| 3-3-1 | Name of depositary | KCTC Korean Collection for Type Cultures |
| | institution | |
| 3-3-2 | Address of depositary | Korean Collection for Type Cultures (KCTC) |
| | institution | 181 Ipsin-gil Jeongeup-si Jeollabuk-do 56212 |
| | | Republic of Korea |
| 3-3-3 | Date of deposit | 26 Feb. 2001 (26 Feb. 2001) |
| 3-3-4 | Accession Number | KCTC 10876BP |
| 3-4 | Additional Indications | |
| 3-5 | Designated States for | All designations |
| | Which Indications are | |
| | Made | |
| 3-6 | Separate Furnishing of | |
| | Indications | |
| | These indications will be | |
| | submitted to the | |
| | International Bureau later | |

FOR RECEIVING OFFICE USE ONLY

| | |
|---|---|
| 0-4 | This form was received |
| | with the international |
| | application: |
| | (yes or no) |
| 0-4-1 | Authorized officer |

FOR INTERNATIONAL BUREAU USE ONLY

| | |
|---|---|
| 0-5 | This form was received by |
| | the international Bureau |
| | on: |
| 0-5-1 | Authorized officer |

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

1. A composition for radiation shield comprising one or more microorganisms selected from the group consisting of Cladosporium sp. microorganism, Phanerochaete sp. microorganism and Trichosporon sp. microorganism.

2. The composition for radiation shield according to claim 1, wherein the Cladosporium sp. microorganism is Cladosporium cladosporioides.

3. The composition for radiation shield according to claim 1, wherein the Phanerochaete sp. microorganism is Phanerochaete chrysosporium or Phanerochaete sordida.

4. The composition for radiation shield according to claim 1, wherein the Trichosporon sp. microorganism is Trichosporon loubieri.

5. A radiation shield material comprising the composition for radiation shield according to claim 1.

6. The radiation shield material according to claim 5, wherein the radiation shield material includes a microorganism immobilized media.

7. The radiation shield material according to claim 6, wherein the microorganism immobilized media is a porous material.

8. The radiation shield material according to claim 6, wherein the microorganism immobilized media is selected from polymer resin, activated carbon, zeolite, non-woven fabric or woven fabric.

9. The radiation shield material according to claim 5, wherein the radiation shield material further includes carbon nanotubes or graphene.
